(19) United States
(12) Patent Application Publication
(10) Pub. No.: US 2019/0203158 A1
(43) Pub. Date: Jul. 4, 2019

(54) DETERGENT COMPOSITION, USE OF DETERGENT COMPOSITION AND A METHOD FOR LAUNDERING A TEXTILE

(71) Applicant: NOVOZYMES A/S, Bagsvaerd (DK)

(72) Inventor: Yuan Xu, Beijing (CN)

(73) Assignee: NOVOZYMES A/S, Bagsvaerd (DK)

(21) Appl. No.: 16/330,246

(22) PCT Filed: Sep. 12, 2017
(86) PCT No.: PCT/CN2017/101366

§ 371 Date: Mar. 4, 2019

(30) Foreign Application Priority Data
Sep. 13, 2016 (CN) PCT/CN2016/098800

(51) Int. Cl. C11D 3/386; C11D 3/04; C11D 3/20; C11D 11/00

(52) U.S. Cl. CPC C11D 3/38627; C11D 3/046; C12N 9/20; C11D 3/2075; C11D 11/0017; C11D 3/2093

(57) ABSTRACT

Disclosed are use of a liquid detergent composition for applying on a textile, a method for laundering a textile and a liquid detergent composition.

### REFERENCE TO A SEQUENCE LISTING

This application contains a Sequence Listing in computer readable form, which is incorporated herein by reference.

### FIELD OF THE INVENTION

Then invention concerns use of a liquid detergent composition for applying on a textile, a method for laundering a textile and a liquid detergent composition.

### DESCRIPTION OF THE RELATED ART

Enzymes have been used within the detergent industry as part of washing formulations for many decades. Lipases are important biocatalysts which have shown to be useful for various applications and a large number of different lipases have been identified and many commercialized.

Lipases have been employed in compositions for the removal of lipid stains by hydrolyzing triglycerides to generate fatty acids. Current cleaning and/or fabric care compositions comprise many active ingredients which are interfering with the ability of lipases to remove lipid stains.

Consumers often experience that stains present on textiles such as chothes, linen or the like may be stubborn and difficult to remove during a normal washing procedure. The consumers handle this by either soaking the textile, apply liquid detergent composition directly on the textile or by using a specific pre-spoting product before washing the textile.

### SUMMARY OF THE INVENTION

The invention concerns use of a liquid detergent composition for applying on a textile, wherein the liquid composition comprises
a. a lipase in an amount of 0.025-0.6 mg enzyme protein/gram detergent composition,
b. a surfactant,
c. a salt comprising a cation and a halogen or a cation and an organic anion, wherein the cation is monovalent, divalent or trivalent and the salt is present in a concentration in the range of 0.00005-0.004 mol/gram detergent composition, wherein ratio of lipase (mg enzyme protein/gram detergent composition) relative to water (g water/gram detergent composition) is in the range of 1:167 to 1:38000.

The invention further concerns a method for laundering a textile, comprising:
a. Applying the liquid detergent composition of the invention on the textile,
b. Leaving the detergent composition on the textile for less than 12 hours,
c. washing the textile in a wash liquor comprising the liquid detergent composition of the invention; and
d. Optionally rinsing the textile.

And the invention concerns a liquid detergent composition comprising:
a. a lipase in an amount of 0.025-0.6 mg enzyme protein/gram detergent composition,
b. a surfactant,
c. a salt comprising a cation and a halogen or a cation and an organic anion, wherein the cation is monovalent, divalent or trivalent and the salt is present in a concentration in the range of 0.00005-0.004 mol/gram detergent composition,

wherein ratio of lipase (mg enzyme protein/gram detergent composition) relative to water (g water/gram detergent composition) is in the range of 1:167 to 1:38000.

### OVERVIEW OF SEQUENCES LISTING

SEQ ID NO: 1 is amino acid sequence of wild-type Thermomyces lanuginosus (previously named Humicola lanuginosa) lipase.

SEQ ID NO: 2 is the amino acid sequence of wild-type Thermomyces lanuginosus lipase with substitutions T231 R+N233R.

### DEFINITIONS

The term “additional enzymes” as used herein, refers to a second or a further enzyme to be included in the detergent composition. The additional enzyme may be any enzyme of another enzyme class than lipase, but it may also comprise a second/further lipase, i.e. the composition according to the invention comprises more than one lipase, such as two or more lipases.

Detergent components: The term “detergent components” is defined herein to mean the types of chemicals which can be used in detergent compositions. Examples of detergent components are alkalis, surfactants, hydrotropes, builders, co-builders, chelators or chelating agents, bleaching system or bleach components, polymers, fabric hueing agents, fabric conditioners, foam boosters, suds suppressors, dispersants, dye transfer inhibitors, fluorescent whitening agents, perfume, optical brighteners, bactericides, fungicides, soil suspending agents, soil release polymers, anti-redeposition agents, enzyme inhibitors or stabilizers, enzyme activators, antioxidants and solubilizers.

Detergent composition: The term “detergent composition” refers to compositions that find use in the removal of undesired compounds from textiles to be cleaned, such as textiles. The detergent composition may be used to e.g. clean textiles for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; fabric fresheners; fabric softeners; and textile and laundry pre-spotters/pretreatment). In addition to containing the enzyme of the invention, the detergent formulation may contain one or more additional enzymes (such as proteases, amylases, lipases, cutinases, cellulases, endoglucanases, xyloglucanases, pectinases, pectin lyases, xanthanases, peroxidaes, haloperoxygenases, catalases and mannanases, or any mixture thereof), and/or detergent components such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

Enzyme Detergency benefit: The term “enzyme detergency benefit” is defined herein as the advantageous effect an enzyme may add to a detergent compared to the same detergent without the enzyme. Important detergency benefits which can be provided by enzymes are stain removal with no or very little visible soils after washing and/or cleaning, prevention or reduction of redeposition of soils released in the washing process (an effect that also is termed anti-redeposition), restoring fully or partly the whiteness of textiles which originally were white but after repeated use and wash have obtained a greyish or yellowish appearance (an effect that also is termed whitening). Textile care benefits, which are not directly related to catalytic stain removal or prevention of redeposition of soils, are also important for enzyme detergency benefits. Examples of such textile care benefits are prevention or reduction of dye transfer from one fabric to another fabric or another part of the same fabric (an effect that is also termed dye transfer inhibition or anti-backstaining), removal of protruding or broken fibers from a fabric surface to decrease pilling tendencies or remove already existing pills or fuzz (an effect that also is termed anti-pilling), improvement of the fabric-softness, colour clarification of the fabric and removal of particulate soils which are trapped in the fibers of the fabric or garment. Enzymatic bleaching is a further enzyme detergency benefit where the catalytic activity generally is used to catalyze the formation of bleaching components such as hydrogen peroxide or other peroxides.

The term “lipase”, “lipase enzyme”, “lipolytic enzyme”, “lipid esterase”, “lipolytic polypeptide”, and “lipolytic protein” as used herein, refers to an enzyme in class EC 3.1,1 as defined by Enzyme Nomenclature. It may have lipase activity (triacylglycerol lipase, EC 3.1.1.3), cutinase activity (EC 3.1.1.74), sterol esterase activity (EC 3.1.1.13) and/or wax-ester hydrolase activity (EC 3.1.1.50). For purposes of the present invention, lipase activity is activity of the refolded lipase and determined according to the procedure described Assay II (Lipase assay: Hydrolytic activity on fatty acids pNP esters). In one aspect, the polypeptide used in accordance with the present invention has at least 20%, e.g., at least 25%, at least 30%, at least 35%, at least 40%, at least 45%, at least 50%, at least 55%, at least 60%, at least 65%, at least 70%, at least 80%, at least 85%, at least 90%, at least 95%, or at least 100% of the lipase activity of the polypeptide of SEQ ID NO: 1.

The term “parent” or “parent lipase” as used herein, refers to a lipase to which a substitution is made to produce the lipase variants of the present invention. The parent may be a naturally occurring (wild-type) polypeptide or a variant or fragment thereof.

The term “corresponding to” as used herein, refers to a way of determining the specific amino acid of a sequence wherein reference is made to a specific amino acid sequence. e.g. for the purposes of the present invention, when references are made to specific amino acid positions, the skilled person would be able to align another amino acid sequence to said amino acid sequence that reference has been made to, in order to determine which specific amino acid may be of interest in said another amino acid sequence. Alignment of another amino acid sequence with e.g. the sequence as set forth in SEQ ID NO: 1, or any other sequence listed herein, has been described elsewhere herein. Alternative alignment methods may be used, and are well-known for the skilled person in the art.

The term “sequence identity” as used herein, refers to the relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used may be gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignmen−Total Number of Gaps in Alignment)

Alternatively, the parameters used may be gap open penalty of 10, gap extension penalty of 0.5, and the EDNAFULL (EMBOSS version of NCBI NUC4.4) substitution matrix. The output of

Needle labeled “longest identity” (obtained using the -nobrief option) is used as the percent identity and is calculated as follows:

(Identical Deoxyribonucleotides×100)/(Length of Alignment−Total Number of Gaps in Alignment)

The term “surfactant” as used herein, refers to any conventional understanding of a surfactant within the art, which may be anionic and/or non-ionic and/or semi-polar and/or zwitterionic and/or cationic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant may preferably be present in concentrations such that it exists mainly in micellar form, i.e. at concentrations above the critical micelle concentration in the main wash. The term “critical micelle concentration” as used herein, refers to a micelle concentration of a surfactant or blend of surfactants is determined by a number of factors such as pH, temperature, ionic strength etc. To determine whether a surfactant or blend of surfactants is present in the main wash solution in concentrations at or above the critical micelle concentration a number of different methods may be used.

Textile: The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used it is intended to include the broader term textiles as well. In the context of the present invention, the term “textile” also covers fabrics.

Variant: The term “variant” means a polypeptide having same type of activity as the parent enzyme but which comprises an alteration, i.e., a substitution, insertion, and/or deletion, at one or more (e.g., several) positions compared to the parent enzyme. A substitution means replacement of the amino acid occupying a position with a different amino acid; a deletion means removal of the amino acid occupying a position; and an insertion means adding an amino acid adjacent to and immediately following the amino acid occupying a position. In the context of the present invention, a variant of an identified lipase has the enzymatic activity of the parent, i.e. the capacity of catalyzing the hydrolytic cleavage of phosphodiester linkages in the DNA backbone (deoxyribonuclease activity).

Wash liquor: The term “wash liquor” is intended to mean the solution or mixture of water and at least a surfactant, optionally including other detergent components e.g. enzymes other than the polypeptide having lipase activity and which is used for laundrering textiles.

The term “improved wash performance” as used herein, refers to a detergent compositions ability to facilitate removal of lipid or lipid-containing stains present on the textile to be cleaned.

### Conventions for Designation of Variants

For purposes of the present invention, the polypeptides disclosed in SEQ ID NO: 1 or 2 may be used to determine the corresponding amino acid residue in another polypeptide. The amino acid sequence of another polypeptide is aligned with the polypeptide disclosed in SEQ ID NO: 1 or 2 depending on whether it is a lipase, an alpha-amylase, or a protease, and based on the alignment, the amino acid position number corresponding to any amino acid residue in the polypeptide disclosed in SEQ ID NO: 1 or 2 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et aL, 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another enzyme may be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et aL, 2005, Nucleic Acids Research 33: 511-518; Katoh and Toh, 2007, Bioinformatics 23: 372-374; Katoh et aL, 2009, Methods in Molecular Biology 537: 39-64; Katoh and Toh, 2010, Bioinformatics 26: 1899-1900), and EMBOSS EMMA employing ClustalW (1.83 or later; Thompson et aL, 1994, Nucleic Acids Research 22: 4673-4680), using their respective default parameters.

When the other enzyme has diverged from the polypeptide of SEQ ID NO: 1, 2, 3, 4, 5, or 6 such that traditional sequence-based comparison fails to detect their relationship (Lindahl and Elofsson, 2000, J. Mol. BioL 295: 613-615), other pairwise sequence comparison algorithms may be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et aL, 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example the SCOP super families of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

It is within the knowledge of the skilled person to determine which alignment tool to use when corresponding amino acid positions must be identified. Therefore, it is contemplated that any available alignment tool that the skilled person find suitable may be used in the context of the present invention.

In describing the enzyme variants described herein, the nomenclature described below is adapted for ease of reference. The accepted IUPAC single letter or three letters amino acid abbreviations are employed. Amino acid positions are indicated with H1, G109, etc. Variants described herein comprises one or more modifications as compared to the parent polypeptide. Accordingly, variants may comprise conservative modifications, in particular, such conservative modifications may be conservative substitutions. Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Asn/Gln, Thr/Ser, Ala/Gly, Ala/Thr, Ser/Asn, Ala/Val, Ser/Gly, Tyr/Phe, Ala/Pro, Lys/Arg, Asp/Asn, Glu/Gln, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Substitutions: For an amino acid substitution, the following nomenclature is used: Original amino acid, position, substituted amino acid. Accordingly, the substitution of glycine at position G109 with alanine is designated as “Gly109Ala” or “G109A”. Multiple mutations are separated by addition marks (“+”) or by commas (“,”), e.g., “Gly109Ala+Leu173Pro” or “G109A,L173P”, representing substitutions at positions 109 and 173 of glysine (G) with alanine (A) and leucine (L) with proline (P), respectively. If more than one amino acid may be substituted in a given position these are listed or divided by slash, such as /. Thus, if both Ala and Pro may be substituted instead of the amino acid occupying at position 109 this is indicated as X109A/P where the X in the present example indicates that different enzymes may be parent e.g. such as an alpha-amylase with SEQ ID NO: 1 or an alpha-amylase having at least 75% identity hereto. Thus, in some cases the variants are represented as 109A/P or X109A/P indicating that the amino acids to be substituted vary depending on the parent enzyme.

Deletions: For an amino acid deletion, the following nomenclature is used: Original amino acid, position, *. Accordingly, the deletion of arginie at position 181 is designated as “Arg181*” or “R181*”. Multiple deletions are separated by addition marks (“+”) or commas, e.g., “Arg181*+Glyl82*” or “R181*+G182*” or “R181*, G182*”.

Insertions: The insertion of an additional amino acid residue such as e.g. a lysine after G#₁ may be indicated by: Gly#₁GlyLys or G#₁GK. Alternatively insertion of an additional amino acid residue such as lysine after G109 may be indicated by: *109aL. When more than one amino acid residue is inserted, such as e.g. a Lys, and Ala after 109 this may be indicated as: Gly109GlyLysAla or G109GKA. In such cases, the inserted amino acid residue(s) may also be numbered by the addition of lower case letters to the position number of the amino acid residue preceding the inserted amino acid residue(s), in this example: *109aK *109bA.

Collectively, substitutions, deletions, and insertions may herein termed “modifications”. Thus, it is to be understood that any variant described herein comprises modifications, such as substitutions, deletions and/or insertions unless otherwise indicated by context.

Multiple modifications: Variants comprising multiple modifications are separated by addition marks (“+”), slash marks (“/”), or by commas (“,”), e.g., “Gly109Pro+Lys391Ala” or “G109P, K391A” representing a substitution of glysine at position 109 and lysine at position 391 with proline and alanine, respectively as described above.

Different modifications: Where different modifications can be introduced at a position, the different modifications are separated by a division (“/”), or by a comma (“,”), e.g., “Gly109Pro,Lys” or “G109P,K” represents a substitution of glysine at position 109 with proline or lysine. Thus, “Gly109Pro,Lys +Lys391Ala” designates the following variants: “Gly109Pro+Lys391Ala”, “Gly109Lys+Lys391Ala” or “G109P,K +K391A”.

### DETAILED DESCRIPTION OF THE INVENTION

The inventor has found that by using a specific liquid detergent compostion for applying on textiles with stubborn stains such as fat-containing stains, the stains are more easily released and removed from the textile during the subsequent washing procedure. A further advantage with the present invention is that the same detergent composition can be used for applying on the textile before wash and be used for the washing process.

The invention concerns use of a liquid detergent composition for applying on a textile, wherein the liquid composition comprises
a. a lipase in an amount of 0.025-0.6 mg enzyme protein/gram detergent composition,
b. a surfactant,
c. a salt comprising a cation and a halogen or a cation and an organic anion, wherein the cation is monovalent, divalent or trivalent and the salt is present in a concentration in the range of 0.00005-0.004 mol/gram detergent composition,

wherein ratio of lipase (mg enzyme protein/gram detergent composition) relative to water (g water/gram detergent composition) is in the range of 1:167 to 1:38000.

The invention further concerns a method for laundering a textile, comprising:
a. Applying the liquid detergent composition of the invention on the textile,
b. Leaving the detergent composition on the textile for less than 12 hours,
c. washing the textile in a wash liquor comprising the liquid detergent composition of the invention; and
d. Optionally rinsing the textile.

And furher the invention concerns a liquid detergent composition comprising:
a. a lipase in an amount of 0.025-0.6 mg enzyme protein/gram detergent composition,
b. a surfactant,
c. a salt comprising a cation and a halogen or a cation and an organic anion, wherein the cation is monovalent, divalent or trivalent and the salt is present in a concentration in the range of 0.00005-0.004 mol/gram detergent composition,

wherein ratio of lipase (mg enzyme protein/gram detergent composition) relative to water (g water/gram detergent composition) is in the range of 1:167 to 1:38000.

Consumers often experience that stains present on textiles such as chothes, linen or the like may be stubborn and difficult to remove during a normal washing procedure. The consumers handle this by either soaking the textile, apply liquid detergent composition directly on the textile or by using a specific pre-spot product before washing the textile. The inventor has found that the same detergent composition can be used for applying on the textile before wash and for the washing when the detergent composition used has a specific concentration of salt and a specific ratio between the lipase and the water content of the detergent composition.

The presence of salt in the detergent composition used is important. When the salt is present in a concentration in the range of 0.00005-0.004 mol/gram detergent composition the best result on stubborn stains is achieved. The concentration of salt is in the range of 0.0001-0.004 mol/gram detergent composition, in the range of 0.00015-0.004 mol/gram detergent composition, in the range of 0.0002-0.003 mol/gram detergent composition, in the range of 0.0003-0.003 mol/gram detergent composition, in the range of 0.0004-0.002 mol/gram detergent composition, in the range of 0.0005-0.001 mol/gram detergent composition or in the range of 0.0005-0.001 mol/gram detergent composition.

Further the amount of lipase in the detergent composition and the ratio between lipase and water in the detergent composition is of importance. The concentration of the lipase should in the range of 0.025-0.6 mg enzyme protein/gram detergent composition and the ration between the lipase and water should be in the range of 1:167 to 1:38000. In one embodiment of the invention the ratio of lipase (mg enzyme protein/gram detergent composition) relative to water (g/gram detergent composition) is in the range of 1:200-1:24000, in the range of 1:250 to 1:22500, in the range of 1:333 to 1:20000, in the range of 1:500 to 1:17500, in the range of 1:1000 to 1:15000, in the range of 1:2000 to 1:12500, in the range of 1:3000 to 1:10000, in the range of 1:4000 to 1:10000 or in the range of 1:5000 to 1:7500.

In addition hereto a surfactant should be present in the detergent composition. The amount of surfactant is in the range of 0.1-0.6 g/gram detergent composition, such as in the range of 0.1-0.5 g/gram detergent composition, in the range of 0.1-0.4 g/gram detergent composition, in the range of 0.1-0.3 g/gram detergent composition, in the range of .01-.02 g/gram detergent composition or in the range of 0.15-0.2 g/gram detergent composition.

The salt present in the detergent composition comprises a cation and a halogen or a cation and an organic anion, wherein the cation is monovalent, divalent or trivalent. The monovalent, divalent or trivalent cations are selected from the group consisting of sodium, potassium, magnesium, calcium, boron, aluminium, iron, cupper, zinc and/or a mixture thereof. In a preferred embodiment of the invention the cations are selected from sodium, calcium and potassium. The organic anions are selected from the group consisting of formate, acetate, citrate, carbonate, hydrogen carbonate and lactate.

In one embodiment the salt is sodium citrate, sodium chloride, sodium carbonate, potassium chloride, calcium chloride or a mixture thereof.

The detergent composition may comprise further enzymes, which may contribute to releasing stains present on the textile. The enzymes can be selected from the group consisting of hemicellulases, peroxidases, proteases, cellulases, xylanases, an additional lipase, phospholipases, esterases, cutinases, pectinases, mannanases, pectate lyases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, DNase chlorophyllases, amylases, perhydrolases, peroxidases, xanthanase and mixtures thereof.

The textile may be any textile material or any structure. The textile can be cellulose based such as natural cellulosics, including cotton, flax/linen, jute, ramie, sisal or coir or manmade cellulosics (e.g. originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). In one embodiment of the invention the the textile comprises cotton and/or polyester. In one embodiment the textile consist of cotton or consist of cotton and/or polyester.

The detergent composition of the invention comprise a lipase. Lipase enzymes are enzymes that degrade fats and oils. In one embodiment of the invention the detergent composition is used for releasing and/or removing lipid stains. Especially lipid stains that has a high content of saturated fat and a high melting point are difficult to remove. The inventor has found that using the present detergent composition on a textile and then washing the textile are superior in removing lipid stains comprising fat with at least 35% saturated fat and having a melting point in the range of 25-50° C. In one embodiment the stains comprise fat having at least 40% saturated fat such as at least 41% saturated fat, at least 42% saturated fat, at least 43% saturated fat, at least 44% saturated fat or at least 45% saturated fat.

The lipid stain comprises vegetable oil and/or animal fat. The vegetable oil can be selected from the group consisting of olive oil, rapeseed oil, soybean oil, canola oil, corn oil, sunflower oil, safflower oil, peanut oil, coconut oil, sesame oil and palm oil. The aninmal fat can be selected from the group consisting of milkfat, lard, tallow, suet, chicken fat, goose fat and/or mixtures thereof.

According to the inventive use and method the detergent composition is applied on the textile and the detergent composition is left on the textile and exposed to the composition for a period of less than 12 hours before the textile is washed. In one embodiment of the invention the textile is exposed to the detergent composition for a period of less than 10 hours, less than 8 hours, less than 6 hours, less than 4 hours, less than 2 hours or less than one hour before the textile is washed. In a preferred embodiment of the invention the textile is exposed to the detergent composition for a period of 5-50 minutes, 5-40 minutes, 5-30 minutes, 5-20 minutes, 5-15 minutes, 5-10 minutes, 5-9 minutes, 5-8 minutes, 5-7 minutes or for a period of 5-6 minutes before the textile is washed.

The wash performance can be expressed as a delta remission value (ARem) as described in Assay I, where the effect of the detergent composition is calculated by taking the measurements from washed swatches with a specific detergent composition and subtract with the measurements from washed same detergent composition without the specific salt and/or enzyme addition for each stain. The total washing performance is calculated as the sum of individual ARem.

The delta remission value when using the detergent composition according to the invention is above 15 when measured with Assay I. In one embodiment the delta remission value is above 20 when measured with Assay I, such as above 25, above 30, above 35 or above 40 when measured with Assay I.

In one embodiment of the invention the composition comprise a parent lipase having at least 80% at least 85%, at least 90%, at least 95% identity, at least 96%, at least 97%, at least 98%, or at least 99% sequence identity to SEQ ID NO: 1 or at least 80% sequence identity to SEQ ID NO: 2. In one embodiment of the invention the lipase has 100% identity to SEQ ID NO: 1. In one embodiment of the invention the lipase has 100% identity to SEQ ID NO: 2.

The lipase can be a lipase variand, which is a variant of a parent lipase and is selected from the group consisting of:
a. variants having at least 80%, at least 85%, at least 90%, at least 95% identity, at least 96%, at least 97%, at least 98%, or at least 99%, but less than 100%, sequence identity to SEQ ID NO: 1;
b. variants having at least 80%, at least 85%, at least 90%, at least 95% identity, at least 96%, at least 97%, at least 98%, or at least 99%, but less than 100%, sequence identity to SEQ ID NO: 2.
   The lipase variant can be a lipase which is a variant of a parent lipase, which variant has lipase activity, has at least 80% but less than 100% sequence identity with SEQ ID NO: 2, and comprises optionally one or more of the following substitutions D96E, D111A, D254S, G163K, P256T, G91T and G38A wherein numbering is according to SEQ ID NO: 2. In one embodiment of the invention the lipase variant further comprise substitutions at positions corresponding to D27R and/or N33Q of SEQ ID NO: 2. In one embodiment of the invention the variant comprises the following substitutions D27R, G38A, D96E, D111A, G163K, D254S and P256T using SEQ ID NO: 2 for numbering.

### Detergent Compositions

In one embodiment, the invention is directed to detergent compositions comprising an a lipase in combination with one or more additional detergent composition components. The choice of additional components is within the skill of the artisan and includes conventional ingredients, including the exemplary non-limiting components set forth below.

### Surfactants

The detergent composition comprises one or more surfactants, of which at least one surfactant is anionic. Other surfactants may be anionic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and may include any conventional surfactant(s) known in the art.

When included therein, the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis (sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

When included therein, the detergent will usually contain from about 0.2% to about 40% by weight of a nonionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein, the detergent will usually contain from about 0% to about 40% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis (2-hydroxyethyl)amine oxide, and combinations thereof.

When included therein, the detergent will usually contain from about 0% to about 40% by weight of a zwitterionic surfactant. Non-limiting examples of zwitterionic surfactants include betaines such as alkyldimethylbetaines, sulfobetaines, and combinations thereof.

### Builders and Co-Builders

The detergent composition may contain about 0-65% by weight, such as about 5% to about 50% of a detergent builder or co-builder, or a mixture thereof. The builder and/or co-builder may particularly be a chelating agent that forms water-soluble complexes with Ca and Mg. Any builder and/or co-builder known in the art for use in detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain 0-50% by weight, such as about 5% to about 30%, of a detergent co-builder. The detergent composition may include include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl) iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), a-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA) , taurine-N,N-diacetic acid (TUDA) and sulfomethyl-N,N-diacetic acid (SMDA), N-(2-hydroxyethypethylenediamine-N,N′,N″-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053.

### Zeolites

A preferred class of zeolites is characterized as“intermediate”silicate/aluminate zeolites. The intermediate zeolites are characterized by SiOx/A10z molar ratios of less than about 10. Preferably the molar ratio of SiO2/A102 ranges from about 2 to about 10. The intermediate zeolites can have an advantage over the “high”zeolites. The intermediate zeolites have a higher affinity for amine-type odors, they are more weight efficient for odor absorption because they have a larger surface area, and they are more moisture tolerant and retain more of their odor absorbing capacity in water than the high zeolites. A wide variety of intermediate zeolites suitable for use herein are commercially available as Valfor® CP301-68, Valfor® 300-63, Valfor® CP300-35, and Valfor®CP300-56, available from PQ Corporation, and the CBV100® series of zeolites from Conteka.

Zeolite materials marketed under the trade name Absents® and Smellrite®, available from The Union Carbide Corporation and UOP are also preferred. Such materials are preferred over the intermediate zeolites for control of sulfur-containing odors, e.g. thiols, mercaptans. When zeolites are used as odor control agents in compositions that are to be sprayed onto surfaces, the zeolite material preferably has a particle size of less than about 10 microns and is present in the composition at a level of less than about 1% by weight of the composition.

### Bleaching Systems

The detergent may contain 0-30% by weight, such as about 1% to about 20%, of a bleaching system. Any bleaching system known in the art for use in detergents may be utilized. Suitable bleaching system components include bleaching catalysts, photobleaches, bleach activators, sources of hydrogen peroxide such as sodium percarbonate, sodium perborates and hydrogen peroxide—urea (1:1), preformed peracids and mixtures thereof. Suitable preformed peracids include, but are not limited to, peroxycarboxylic acids and salts, diperoxydicarboxylic acids, perimidic acids and salts, peroxymonosulfuric acids and salts, for example, Oxone (R), and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with hydrogen peroxide to form a peracid via perhydrolysis. The peracid thus formed constitutes the activated bleach. Suitable bleach activators to be used herein include those belonging to the class of esters, amides, imides or anhydrides. Suitable examples are tetraacetylethylenediamine (TAED), sodium 4-[(3,5,5-trimethylhexanoy)oxy]benzene-1-sulfonate (ISONOBS), 4-(dodecanoyloxy)benzene-1-sulfonate (LOBS), 4-(decanoyloxy)benzene-1-sulfonate, 4-(decanoyloxy)benzoate (DOBS or DOBA), 4-(nonanoyloxy)benzene-1-sulfonate (NOBS), and/or those disclosed in WO 98/17767. A particular family of bleach activators of interest was disclosed in EP624154 and particulary preferred in that family is acetyl triethyl citrate (ATC). ATC or a short chain triglyceride like triacetin has the advantage that it is environmentally friendly Furthermore acetyl triethyl citrate and triacetin have good hydrolytical stability in the product upon storage and are efficient bleach activators. Finally ATC is multifunctional, as the citrate released in the perhydrolysis reaction may function as a builder. Alternatively, the bleaching system may comprise peroxyacids of, for example, the amide, imide, or sulfone type. The bleaching system may also comprise peracids such as 6-(phthalimido) peroxyhexanoic acid (PAP). The bleaching system may also include a bleach catalyst. In some embodiments the bleach component may be an organic catalyst selected from the group consisting of organic catalysts having the following formulae:

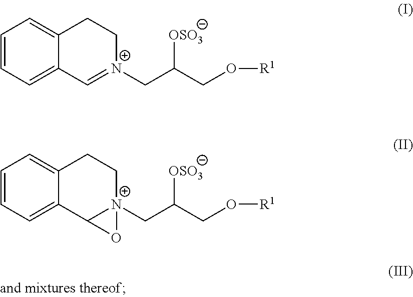

wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl. Other exemplary bleaching systems are described, e.g. in WO2007/087258, WO2007/087244, WO2007/087259, EP1867708 (Vitamin K) and WO2007/087242. Suitable photobleaches may for example be sulfonated zinc or aluminium phthalocyanines.

Preferably, the bleach component comprises a source of peracid in addition to bleach catalyst, particularly organic bleach catalyst. The source of peracid may be selected from (a) preformed peracid; (b) percarbonate, perborate or persulfate salt (hydrogen peroxide source) preferably in combination with a bleach activator; and (c) perhydrolase enzyme and an ester for forming peracid in situ in the presence of water in a textile treatment step.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO 2005/03274, WO 2005/03275, WO 2005/03276 and EP 1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition

WO 2018/050047 PCT/CN2017/101366 may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO 2007/087243.

Enzymes

The detergent additive as well as the detergent composition may comprise one or more additional enzymes such as hemicellulases, peroxidases, proteases, cellulases, xylanases, an lipase, phospholipases, esterases, cutinases, pectinases, mannanases, pectate lyases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, DNase chlorophyllases, amylases, perhydrolases, peroxidases, xanthanase and mixtures thereof.

In general the properties of the selected enzyme(s) should be compatible with the selected detergent, (La, pH-optimum, compatibility with other enzymatic and non-enzymatic ingredients, etc.), and the enzyme(s) should be present in effective amounts.

### Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and WO99/001544.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Celluzyme™, and Carezyme™ (Novozymes A/S) Carezyme Premium™ (Novozymes A/S), Celluclean ™ (Novozymes A/S), Celluclean Classic™ (Novozymes A/S), Cellusoft™ (Novozymes A/S), Whitezyme™ (Novozymes A/S), Clazinase™, and Puradax HA™ (Genencor International Inc.), and KAC-500(B)™ (Kao Corporation).

### Mannanases

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes A/S).

### Cellulase

Suitable cellulases include complete cellulases or mono-component endoglucanases of bacterial or fungal origin. Chemically or genetically modified mutants are included. The cellulase may for example be a mono-component or a mixture of mono-component endo-1,4-beta-glucanase often just termed endoglucanases. Suitable cellulases include a fungal cellulase from Humicola insolens (U.S. Pat. No. 4,435,307) or from Trichoderma, e.g. T. reesei or T. viride. Examples of cellulases are described in EP 0 495 257. Other suitable cellulases are from Thielavia e.g. Thielavia terrestris as described in WO 96/29397 or Fusarium oxysporum as described in WO 91/17244 or from Bacillus as described in, WO 02/099091 and JP 2000210081. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 Commercially available cellulases include Carezyme®, Celluzyme®, Celluclean®, Celluclast® and Endolase®; Renozyme®; Whitezyme® (Novozymes A/S) Puradax®, Puradax HA, and Puradax EG (available from Genencor).

### Peroxidases/Oxidases

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme™ (Novozymes A/S).

### Proteases

Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases derived from Cellumonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those derived from Bacillus amyloliquefaciens.

Examples of useful proteases are the variants described in: WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 24, 27, 42, 55, 59, 60, 66, 74, 85, 96, 97, 98, 99, 100, 101, 102, 104, 116, 118, 121, 126, 127, 128, 154, 156, 157, 158, 161, 164, 176, 179, 182, 185, 188, 189, 193, 198, 199, 200, 203, 206, 211, 212, 216, 218, 226, 229, 230, 239, 246, 255, 256, 268 and 269 wherein the positions correspond to the positions of the Bacillus Lentus protease shown in SEQ ID NO 1 of WO 2016/001449. More preferred the subtilase variants may comprise the mutations: S3T, V41, S9R, S9E, A15T, S24G, S24R, K27R, N42R, S55P, G59E, G59D, N60D, N60E, V66A, N74D, N85S, N85R, G96S, G96A, S97G, S97D, S97A, S97SD, S99E,

S99D, S99G, S99M, S99N, S99R, 599H, S101A, V1021, V102Y, V102N, S104A, G116V, G116R, H118D, H118N, N1205, 5126L, P127Q, S128A, S154D, A156E, G157D, G157P, S158E, Y161A, R164S, Q176E, N179E, S182E, Q185N, A188P, G189E, V193M, N198D, V1991, Y203W, 5206G, L211Q, L211D, N212D, N2125, M2165, A226V, K229L, Q230H, Q239R, N246K, N255W, N255D, N255E, L256E, L256D T268A, R269H. The protease variants are preferably variants of the Bacillus Lentus protease (Savinase®) shown in SEQ ID NO 1 of WO 2016/001449, the Bacillus amylolichenifaciens protease (BPN′) shown in SEQ ID NO 2 of WO2016/001449. The protease variants preferably have at least 80% sequence identity to SEQ ID NO 1 or SEQ ID NO 2 of WO 2016/001449.

A protease variant comprising a substitution at one or more positions corresponding to positions 171, 173, 175, 179, or 180 of SEQ ID NO: 1 of WO2004/067737, wherein said protease variant has a sequence identity of at least 75% but less than 100% to SEQ ID NO: 1 of WO2004/067737.

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Blaze®, Blaze Evity® 100T, Blaze Evity® 125T, Blaze Evity® 150T, Neutrase®, Everlase® and Esperase® (Novozymes A/S), those sold under the tradename Maxatase®, Maxacal®, Maxapem®, Purafect Ox®, Purafect OxP®, Puramax®, FN2®, FN3®, FN4®, Excellase®, Excellenz P1000™, Excellenz P1250™, Eraser®, Preferenz P100™, Purafect Prime®, Preferenz P110™, Effectenz P1000™, Purafect®™, Effectenz P1050™, Purafect Ox®™, Effectenz P2000™, Purafast®, Properase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

### Lipases and Cutinases:

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

### Amylases:

Suitable amylases which can be used together with the detergent composition of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T49I+G107A+H156Y+A181T+N190F+1201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T1311, T1651, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T1311+T1651+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, 1203, S241, R458, T459, D460, G476 and G477. More preferred variants of SEQ ID NO: 1 are those having the substitution in one of more of the following positions: K176L, E187P, N192FYH, M199L, 1203YF, S241QADN, R458N, T459S, D460T, G476K and G477K and/or deletion in position R178 and/or S179 or of T180 and/or G181.

Most preferred amylase variants of SEQ ID NO: 1 are those having the substitutions:

E187P+1203Y+G476K

E187P+1203Y+R458N+T4595+D460T+G476K

wherein the variants optionally further comprises a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, 1181, G182, M200, L204, E242, G477 and G478. More preferred variants of SEQ ID NO: 1 are those having the substitution in one of more of the following positions: N21D, D97N, V1281 K177L, M200L, L204YF, E242QA, G477K and G478K and/or deletion in position R179 and/or S180 or of 1181 and/or G182. Most preferred amylase variants of SEQ ID NO: 1 are those having the substitutions:

N21D+D97N+V128I

wherein the variants optionally further comprises a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™, Stainzyme Plus™, Natalase™, Liquozyme X and BAN™ (from Novozymes A/S), and Rapidase™ Purastar™/Effectenz™, Powerase, Preferenz S1000, Preferenz S100 and Preferenz S110 (from Genencor International Inc./DuPont).

### Peroxidases/Oxidases

A peroxidase according to the invention is a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

A peroxidase also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions.

In an embodiment, the haloperoxidase is a chloroperoxidase. Preferably, the haloperoxidase is a vanadium haloperoxidase, i.e., a vanadate-containing haloperoxidase. In a preferred method of the present invention the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

In an preferred embodiment, the haloperoxidase is derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

An oxidase include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), ora bilirubin oxidase (EC 1.3.3.5). Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus.

A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g. as disclosed in U.S. Pat. No. 4,106,991 and 4,661,452 and may optionally be coated by methods known in the art. Examples of waxy coating materials are polyethyleneglycol (PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

### Adjunct Materials

Any detergent components known in the art for use in laundry detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. The choice of such ingredients is well within the skill of the artisan.

### Dispersants

The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

### Dye Transfer Inhibiting Agents

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

### Fluorescent Whitening Agent

The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

### Soil Release Polymers

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Anti-Redeposition Agents

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

### Rheology Modifiers

The detergent compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids: US2009/0011970 A1.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent , which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent.

A liquid or gel detergent may be non-aqueous.

### Formulation of Enzyme In Co-Granule

The enzyme may according to the invention be formulated as a granule for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme co-granulates for the detergent industry are disclosed in the IP.com disclosure IPCOM000200739D.

Another example of formulation of enzymes by the use of co-granulates are disclosed in WO 2013/188331, which relates to a detergent composition comprising (a) a multi-enzyme co-granule; (b) less than 10 wt zeolite (anhydrous basis); and (c) less than 10 wt phosphate salt (anhydrous basis), wherein said enzyme co-granule comprises from 10 to 98 wt % moisture sink component and the composition additionally comprises from 20 to 80 wt % detergent moisture sink component.

WO 2013/188331 also relates to a method of treating and/or cleaning a surface, preferably a fabric surface comprising the steps of (i) contacting said surface with the detergent composition as claimed and described herein in an aqueous wash liquor, (ii) rinsing and/or drying the surface.

The multi-enzyme co-granule may comprise an enzyme and (a) one or more enzymes selected from the group consisting of first-wash lipases, cleaning cellulases, xyloglucanases, perhydrolases, peroxidases, lipoxygenases, laccases and mixtures thereof; and (b) one or more enzymes selected from the group consisting of hemicellulases, proteases, care cellulases, cellobiose dehydrogenases, xylanases, phospho lipases, esterases, cutinases, pectinases, mannanases, pectate lyases, keratinases, reductases, oxidases, phenoloxidases, ligninases, pullulanases, tannases, pentosanases, lichenases glucanases, arabinosidases, hyaluronidase, chondroitinase, amylases, and mixtures thereof.

### The Present Invention is Descriped in the Following Paragraphs:

1. Use of a liquid detergent composition for applying on a textile, wherein the liquid composition comprises
a. a lipase in an amount of 0.025-0.6 mg enzyme protein/gram detergent composition,
b. a surfactant,
c. a salt comprising a cation and a halogen or a cation and an organic anion, wherein the cation is monovalent, divalent or trivalent and the salt is present in a concentration in the range of 0.00005-0.004 mol/gram detergent composition,
   wherein ratio of lipase (mg enzyme protein/gram detergent composition) relative to water (g water/gram detergent composition) is in the range of 1:167 to 1:38000.
   2. Use according to paragraph 1, wherein the wherein ratio of lipase (mg enzyme protein/gram detergent composition) relative to water (g/gram detergent composition) is in the range of 1:200-1:24000, in the range of 1:250 to 1:22500, in the range of 1:333 to 1:20000, in the range of 1:500 to 1:17500, in the range of 1:1000 to 1:15000, in the range of 1:2000 to 1:12500, in the range of 1:3000 to 1:10000, in the range of 1:4000 to 1:10000 or in the range of 1:5000 to 1:7500.
   3. Use according to any of paragraphs 1-2, wherein the concentration of the salt is in the range of 0.0001-0.004 mol/gram detergent composition, in the range of 0.00015-0.004 mol/gram detergent composition, in the range of 0.0002-0.003 mol/gram detergent composition, in the range of 0.0003-0.003 mol/gram detergent composition, in the range of 0.0004-0.002 mol/gram detergent composition, in the range of 0.0005-0.001 mol/gram detergent composition or in the range of 0.0005-0.001 mol/gram detergent composition.
   4. Use according to any of the preceding paragraphs, wherein the surfactant is in an amount of 0.1-0.6 g/gram detergent composition, such as in the range of 0.1-0.5 g/gram detergent composition, in the range of 0.1-0.4 g/gram detergent composition, in the range of 0.1-0.3 g/gram detergent composition, in the range of 01-0.02 g/gram detergent composition or in the range of 0.15-0.2 g/gram detergent composition.
   5. Use according to any of the preceding paragraphs, wherein the monovalent, divalent or trivalent cations are selected from the group consisting of sodium, potassium, magnesium, calcium, boron, aluminium, iron, cupper, zinc and/or a mixture thereof.
   6. Use according to paragraph 5, wherein the cations are selected from sodium, calcium and potassium.
   7. Use according any of the preceding paragraphs, wherein the organic anion is selected from the group consisting of formate, acetate, citrate, carbonate, hydrogen carbonate and lactate.
   8. Use according to any of the preceding paragraphs, wherein the salt is sodium citrate, sodium chloride, sodium carbonate, potassium chloride, calcium chloride or a mixture thereof.
   9. Use according to any of the preceding paragraphs, wherein the detergent composition further comprises one or more enzymes selected from the group consisting of hemicellulases, peroxidases, proteases, cellulases, xylanases, an additional lipase, phospholipases, esterases, cutinases, pectinases, mannanases, pectate lyases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, DNase chlorophyllases, amylases, perhydrolases, peroxidases, xanthanase and mixtures thereof.
   10. Use according to any of the preceding paragraphs, wherein the textile comprises cotton and/or polyester.
   11. Use according to paragraph 10, wherein the textile consists of cotton and/or polyester.
   12. Use according to any of the preceding paragraphs, wherein the detergent composition is used for releasing and/or removing lipid stains.
   13. Use according to paragraph 12, wherein the lipid stain comprises fat with at least 35% saturated fat and having a melting point in the range of 25-50° C.
   14. Use according to paragraph 13, wherein the fat has at least 40% saturated fat such as at least 41% saturated fat, at least 42% saturated fat, at least 43% saturated fat, at least 44% saturated fat or at least 45% saturated fat.
   15. Use according to any of paragraphs 13-14, wherein the lipid stain comprises vegetable oil and/or animal fat.
   16. Use according to paragraph 15, wherein the vegetable oil can be selected from the group consisting of olive oil, rapeseed oil, soybean oil, canola oil, corn oil, sunflower oil, safflower oil, peanut oil, coconut oil, sesame oil and palm oil.
   17. Use according to paragraph 15, wherein the animal fat can be selected from the group consisting of milkfat, lard, tallow, suet, chicken fat, goose fat and/or mixtures thereof.
   18. Use according to any of the preceding paragraphs, wherein the detergent composition is left on the textile for a period of less than 12 hours.
   19. Use according to paragraph 18, wherein the textile is exposed to the detergent composition for a period of less than 10 hours, less than 8 hours, less than 6 hours, less than 4 hours, less than 2 hours or less than one hour.
   20. Use according to any of paragraphs 18-19, wherein the textile is exposed to the detergent composition for a period of 5-50 minutes, 5-40 minutes, 5-30 minutes, 5-20 minutes, 5-15 minutes, 5-10 minutes, 5-9 minutes, 5-8 minutes, 5-7 minutes or fora period of 5-6 minutes.
   21. Use according to any of the preceding paragraphs, wherein the delta remission value is above 15 when measured with Assay I.
   22. Use according to paragraph 21, wherein the delta remission value is above 20 when measured with Assay I, such as above 25, above 30, above 35 or above 40 when measured with Assay I.
   23. Method for laundering a textile, comprising:
   a. Applying the liquid detergent composition of paragraphs 43-56 on the textile,
   b. Leaving the detergent composition on the textile for less than 12 hours,
   c. Washing the textile in a wash liquor comprising the liquid detergent composition of paragraphs 43-56; and
   d. Optionally rinsing the textile.
   24. Method according to paragraph 23, wherein the textile is rinsed in water optionally comprising a softener.
   25. Method according to any of paragraph 23-24, wherein the textile comprises cotton and/or polyester.
   26. Method according to paragraph 25, wherein the textile consists of cotton and/or polyester.
   27. Method according to any of the preceding method paragraphs, wherein the detergent composition is used for releasing and/or removing lipid stains.
   28. Method according to paragraph 27, wherein the lipid stain comprises fat with at least 35% saturated fat and having a melting point in the range of 25-50° C.
   29 .Method according to paragraph 28, wherein the fat has at least 40% saturated fat such as at least 41% saturated fat, at least 42% saturated fat, at least 43% saturated fat, at least 44% saturated fat or at least 45% saturated fat.
   30. Method according to any of paragraphs 27-29, wherein the lipid stain comprises vegetable oil and/or animal fat.
   31. Method according to paragraph 30, wherein the vegetable oil can be selected from the group consisting of olive oil, rapeseed oil, soybean oil, canola oil, corn oil, sunflower oil, safflower oil, peanut oil, coconut oil, sesame oil and palm oil.
   32. Method according to paragraph 30, wherein the animal fat can be selected from the group consisting of milkfat, lard, tallow, chicken fat, goose fat and/or mixtures thereof.
   33. Method according to any of the preceding method paragraphs, wherein the detergent composition is left on the textile for a period of less than 12 hours.
   34. Method according to paragraph 33, wherein the textile is exposed to the detergent composition for a period of less than 10 hours, less than 8 hours, less than 6 hours, less than 4 hours, less than 2 hours or less than one hour.
   35. Method according to any of paragraphs 33 or 34, wherein the textile is exposed to the detergent composition for a period of 5-50 minutes, 5-40 minutes, 5-30 minutes, 5-20 minutes, 5-15 minutes, 5-10 minutes, 5-9 minutes, 5-8 minutes, 5-7 minutes or fora period of 5-6 minutes.
   36. Method according to any of the preceding method paragraphs, wherein the delta remission value is above 15 when measured with Assay I.
   37. Method according to paragraph 36, wherein the delta remission value is above 20 when measured with Assay I, such as above 25, above 30, above 35 or above 40 when measured with Assay I.
   38. Method according to any of the preceding method paragraphs, wherein the concentration of the lipase in the wash liquor under step c is in the range of 0.03-0.9 mg enzyme protein/g water.
   39. A liquid detergent composition comprising:
   a. a lipase in an amount of 0.025-0.6 mg enzyme protein/gram detergent composition,
   b. a surfactant,
   c. a salt comprising a cation and a halogen or a cation and an organic anion, wherein the cation is monovalent, divalent or trivalent and the salt is present in a concentration in the range of 0.00005-0.004 mol/gram detergent composition,
   wherein ratio of lipase (mg enzyme protein/gram detergent composition) relative to water (g water/gram detergent composition) is in the range of 1:167 to 1:38000.
   40. Composition according to paragraph 39, wherein the ratio of lipase (mg enzyme protein/gram detergent composition) relative to water (g/gram detergent composition) is in the range of 1:200-1:24000, in the range of 1:250 to 1:22500, in the range of 1:333 to 1:20000, in the range of 1:500 to 1:17500, in the range of 1:1000 to 1:15000, in the range of 1:2000 to 1:12500, in the range of 1:3000 to 1:10000, in the range of 1:4000 to 1:10000 or in the range of 1:5000 to 1:7500.
   41. Composition according to any of paragraphs 39-40, wherein the surfactant is in an amount of 0.1-0.6 g/gram detergent composition, such as in the range of 0.1-0.5 g/gram detergent composition, in the range of 0.1-0.4 g/gram detergent composition, in the range of 0.1-0.3 g/gram detergent composition, in the range of .01-.02 g/gram detergent composition or in the range of 0.15-0.2 g/gram detergent composition.
   42. Composition according to any of paragraphs 39-41, wherein the concentration of the salt is in the range of 0.0001-0.004 mol/gram detergent composition, in the range of 0.00015-0.004 mol/gram detergent composition, in the range of 0.0002-0.003 mol/gram detergent composition, in the range of 0.0003-0.003 mol/gram detergent composition, in the range of 0.0004-0.002 mol/gram detergent composition, in the range of 0.0005-0.001 mol/gram detergent composition or in the range of 0.0005-0.001 mol/gram detergent composition.
   43. Composition according any of the preceding composition paragraphs, wherein the monovalent, divalent or trivalent cations are selected from the group consisting of sodium, potassium, magnesium, calcium, boron, aluminium, iron, cupper, zinc and/or a mixture thereof.
   44. Composition according any of the preceding composition paragraphs, wherein the organic anion is selected from the group consisting of formate, acetate, citrate, carbonate, hydrogen carbonate and lactate.
   45. Composition according to paragraph 43, wherein the cations are selected from sodium, calcium and potassium.
   46. Composition according any of the preceding composition paragraphs, wherein the salt is sodium citrate, sodium chloride, sodium carbonate, potassium chloride, calcium chloride or a mixture thereof.
   47. Composition according any of the preceding composition paragraphs, wherein the detergent composition further comprises one or more enzymes selected from the group consisting of hemicellulases, peroxidases, proteases, cellulases, xylanases, an additional lipase, phospholipases, esterases, cutinases, pectinases, mannanases, pectate lyases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, DNase chlorophyllases, amylases, perhydrolases, peroxidases, xanthanase and mixtures thereof.
   48. Composition according to any of the preceding composition paragraphs, wherein the detergent composition is used for releasing and/or removing lipid stains.
   49. Composition according to any of the preceding composition paragraphs, wherein the lipase has at least 80%, at least 85%, at least 90%, at least 95%, at least 96%, at least 97%, at least 98%, at least 99% sequence identity to SEQ ID NO: 1 or at least 80% %, at least 85%, at least 90%, at least 95%, at least 96%, at least 97%, at least 98%, at least 99%sequence identity to SEQ ID NO: 2.
   50. Composition according to paragraph 49, wherein the lipase is a variant of a parent lipase and is selected from the group consisting of:
   a. variants having at least 80%, at least 85%, at least 90%, at least 95% identity, at least 96%, at least 97%, at least 98%, or at least 99%, but less than 100%, sequence identity to SEQ ID NO: 1;
   b. variants having at least 80%, at least 85%, at least 90%, at least 95% identity, at least 96%, at least 97%, at least 98%, or at least 99%, but less than 100%, sequence identity to SEQ ID NO: 2.
   51. Composition according to any of paragraphs 49-50, wherein the lipase is a variant of a parent lipase, which variant has lipase activity, has at least 80% but less than 100% sequence identity with SEQ ID NO: 2, and comprises optionally one or more of the following substitutions D96E, D111A, D254S, G163K, P256T, G91T and G38A wherein numbering is according to SEQ ID NO: 2.
   52. Composition according to paragraph 51, wherein the variant further comprises substitutions at positions corresponding to D27R and/or N33Q wherein numberin is according to SEQ ID NO: 2.
   53. Composition according to paragraph 52, wherein the variant comprises the following substitutions D27R, G38A, D96E, D111A, G163K, D254S and P256T wherein the numbering is according to SEQ ID NO: 2.
   54. Composition according to paragraph 49, wherein the lipase has 100% identity to SEQ ID NO: 1.
   55. Composition according to paragraph 49, wherein the lipase has 100% identity to SEQ ID NO: 2.

### EXAMPLES

### Detergent Compositions

Liby Quanxiao: Surfactants, anti-redeposition agent, builders, perfume, fabric care agent, water.

Bluemoon deepclean: Surfactants, stain removal booster, perfume, water.

Nice Chaoneng: Soap, surfactants, water, softener, builders, perfume, water.

### Washing Methods

### Terg-O-Tometer (TOM) Wash Assay

The Tergo-To-Meter (TOM) is a medium scale model wash system that can be applied to test 16 different wash conditions simultaneously. A TOM is basically a large temperature controlled water bath with up to 16 open metal beakers submerged into it. Each beaker constitutes one small top loader style washing machine and during an experiment, each of them will contain a solution of a specific detergent/enzyme system and the soiled and unsoiled fabrics its performance is tested on. Mechanical stress is achieved by a rotating stirring arm, which stirs the liquid within each beaker. Because the TOM beakers have no lid, it is possible to withdraw samples during a TOM experiment and assay for information on-line during wash.

The TOM model wash system is mainly used in medium scale testing of detergents and enzymes at US or LA/AP wash conditions. In a TOM experiment, factors such as the ballast to soil ratio and the fabric to wash liquor ratio can be varied. Therefore, the TOM provides the link between small scale experiments, such as AMSA and mini-wash, and the more time consuming full scale experiments in top loader washing machines.

Equipment: The water bath with 16 steel beakers and 1 rotating arm per beaker with capacity of 500, 1000 or 1200mL of detergent solution. Temperature ranges from 5 to 55° C. The water bath has to be filled up with deionised water. Rotational speed can be set up to 70 to 200 rpm/min.

Set temperature in the Terg-O-Tometer and start the rotation in the water bath. Wait for the temperature to adjust (tolerance is +/−0.5° C.).

All beakers shall be clean and without traces of prior test material.

Prepare wash solution with temperature and water hardness in a bucket, the detergent composition with/without enzymes is present on the swatch.

Add 1000 ml deionised water and ingredient for adjusting water hardness and optionally detergent composition into a TOM beaker.

Start agitation at 120rpm.

Sprinkle the swatches into the beaker and then the ballast load.

Time measurement start when the swatches and ballast are added to the beaker.

Wash for 20 minutes.

Stop agitation.

Transfer the wash load from TOM beaker to a sieve and rinse with cold tap water.

Separate the soil swatches from the ballast load. The soil swatches are transferred to a 5L beaker with cold tap water under running water. Keep the ballast load separately for the coming inactivation.

Set the timer to 5 minutes.

### Assay I

Wash performance is expressed as a remission value (Rem). After washing and rinsing the swatches were spread out flat and allowed to air dry at room temperature over night. All washes are evaluated the day after the wash. Light reflectance evaluations of the swatches were done using a Macbeth Color Eye 7000 reflectance spectrophotometer with very small aperture. The measurements were made without UV in the incident light and remission at 460 nm was extracted. Measurements were made on swatches washed with a detergent composition and with the same detergent composition with a specific amount of salt and/or lipase added. The test swatch to be measured was placed on top of another swatch of same type and colour (twin swatch). With only one swatch of each kind per beaker, a swatch from a replicate wash was used in this way.

Remission values can either be expressed as the remission value measured or a as the delta remission value. For delta remission values remission values for individual swatches were calculated by subtracting the remission value of the swatches washed with a detergent composition comprising a specific amount of salt and/or lipase added from the remission value of the swatch washed with the same detergent composition but without the specific amount of salt and/or lipase added. The total wash performance for each stained swatch set was calculated as the average individual delta Rem-values.

### Assay II

p-nitrophenyl (pNP) Assay:

The hydrolytic activity of a lipase may be determined by a kinetic assay using p-nitrophenyl acyl esters as substrate.

A 100 mM stock solution in DMSO of the substrates: p-Nitrophenyl butyrate (C4), p-Nitrophenyl caproate (C6), p-Nitrophenyl caprate (C10), p-Nitrophenyl laurate (C12) and p-Nitrophenyl palmitate (C16) (all from Sigma-Aldrich Danmark A/S, Kirkebjerg Allé 84, 2605 Brøndby; Cat.no.: C4:N-9876, C6: N-0502, C10: N-0252, C12: N-2002, C16: N-2752) may be diluted to a final concentration of 1mM 25 into assay buffer (50mM Tris; pH 7.7; 0.4% TritonX-100). The lipase of the invention, the parent lipase and appropriate controls e.g. Buffer (negative), Lipolase™ & Lipex™ (positive) in 50mM Hepes; pH 8.0; 10 ppm TritonX-100; +/−20 mM CaCl₂ may be added to the substrate solution in the following final concentrations: 0.01 mg/ml; 5×10-3 mg/ml; 2.5×10-4 mg/ml; and 1.25×10-4 mg/ml in 96-well NUNC plates (Cat. No:260836, Kamstrupvej 90, DK-4000, Roskilde). Release of p-nitrophenol by hydrolysis of p-nitrophenyl acyl may be monitored at 405nm for 5 minutes in 10 second intervals on a Spectra max 190 (Molecular Devices GmbH, Bismarckring 39, 88400 Biberach an der Riss, GERMANY). The hydrolytic activity towards one or more substrates of a variant may be compared to that of the parent lipase.

### Example 1

### Example of Effect in Full Scale Wash

This is the test method used to test the wash performance of modified detergent compositions in full scale wash under Acian Pacific conditions (washing in a top loader washing machine).

Greasy swatches were pre-treated with different detergent compositions including a salt and lipase content for a certain period of time before they were put into regular full scale wash. After wash, the swatches were dried. Color difference was measured on a MacBeth Color Eye spectrophometer (Assay I).

### Swatch Used:

TABLE 1
Swatch list

| | | Amount used in each |
|---|---|---|
| Producer | Swatch name | wash |
| Center For | NZ-H001 Lard with colorant | 4 pieces |
| Testmaterials | NZ-H002 Lard with colorant | 4 pieces |
| BV, | C-H150 Soya oil with colorant | 2 pieces |
| the Netherlands | C-S-78 Soy bean oil/Pigment | 2 pieces |
| | KC-S-61 Beef fat with colorant | 2 pieces |
| | KC-H-113 Lard, sudan red | 2 pieces |

### Applying Detergent Composition on the Swatches

Pipette 1.0 g of each detergent composition in the table 2 and drop it onto the stain area of each piece of swatch from table 1, which were equally distributed and attached to two pieces of teatowel.
Spread the detergent liquid over the stain area by one finger or a dropper bulb head.
Leave for 10 min before regular full-scale wash.

### Full-Scale Wash Conditions

Temperature: 25° C.
Washing programme: Standard cotton wash without pre-wash: “Cottons”.
Water level 33L.
Water hardness: 14° dH, Ca²⁺:Mg²⁺:HCO₃⁻=2:1:4.5.
Extra detergent composition (original or modified as described in table 2) loaded into the drum: 34 g (resulting in that the total amount of detergent composition in each wash is 50 g/wash, including the amount applied on the 16 swatches. This is close to regular detergent composition load in Chinese household laundry).

### Equipment Used:

Washing machine: Panasonic XQB65-Q680U
Water meters and automatically data collection system
MacBeth Color Eye spectrophotometer
Mettler Toledo analytical balance AB104-S

### For the Preparation and Adjustment of Water Hardness the Following Ingredients are Needed:

Calcium chloride (CaCl₂.2H₂O)

Magnesium chloride (MgCL₂.6H₂O)
Sodium Hydrogen Carbonate (NaHCO₃)

### Ballast

The ballast consists of clean white T-shirt without optical whitener made of cotton. The ballast weight, dryness and item composition must be the same in each wash.

After each wash the ballast is inactivated in an industrial washer at 95° C. /15 min wash without detergent composition.

### Drying Procedure

After wash, swatches were put on tray or hang in line and dry at room temperature. The room has a de-humidifier working for 24 h per day to keep the room dry

### Measurement

Swatches were removed from teatowels, and swatches with colorants are evaluated by measurement of light reflectance difference at 460 nm wave length using a Color Eye as described in Assay I.

TABLE 2
results

| | | Swatch | | | | | |
|---|---|---|---|---|---|---|---|
| | | | | C-H150 | | KC-S-61 | |
| | | NZ-H001 | NZ-H002 | Soya oil | C-S-78 | Beef fat | KC-H-113 |
| | | Lard with | Lard with | with | Soy bean | with | Lard, |
| | Modification | colorant | colorant | colorant | oil/Pigment | colorant | sudan red |
| Original | to detergent | R460/Delta | R460/Delta | R460/Delta | R460/Delta | R460/Delta | R460/Delta |
| detergent | composition | R460 | R460 | R460 | R460 | R460 | R460 |
| Liby | none | 38.02 | 65.45 | 72.36 | 65.14 | 52.13 | 25.05 |
| Quanxiao | | | | | | | |
| Liby | 0.04 mg of | 60.86/ | 78.46/ | 73.08/ | 67.54/ | 57.30/ | 36.51/ |
| Quanxiao | SEQ ID | 22.84 | 13.01 | 0.72 | 2.4 | 5.17 | 11.46 |
| | NO: 2 | | | | | | |
| | variant | | | | | | |
| Liby | 0.08 mg of | 67.84/ | 85.54/ | 73.08/ | 67.57/ | 58.52/ | 38.22/ |
| Quanxiao | SEQ ID | 29.82 | 20.09 | 0.72 | 2.43 | 6.39 | 13.17 |
| | NO: 2 | | | | | | |
| | variant | | | | | | |
| Liby | 5% sodium | 56.10/ | 76.09/ | 72.66/ | 66.07/ | 51.54/ | 38.58/ |
| Quanxiao | citrate | 18.08 | 10.64 | 0.3 | 0.93 | −0.59 | 13.53 |
| Liby | 5% sodium | 81.11/ | 88.05/ | 74.49/ | 69.40/ | 65.15/ | 56.88/ |
| Quanxiao | citrate, | 43.09 | 22.6 | 2.13 | 4.26 | 13.02 | 31.83 |
| | 0.04 mg of | | | | | | |
| | SEQ ID | | | | | | |
| | NO: 2 | | | | | | |
| | variant | | | | | | |
| Liby | 3% sodium | 51.67/ | 74.14/ | 71.65/ | 65.13/ | 51.68/ | 33.74/ |
| Quanxiao | chloride | 13.65 | 8.69 | −0.71 | −0.01 | −0.45 | 8.69 |
| Liby | 3% sodium | 77.56/ | 88.30/ | 74.24/ | 70.33/ | 64.99/ | 55.79/ |
| Quanxiao | chloride, | 39.54 | 22.85 | 1.88 | 5.19 | 12.86 | 30.74 |
| | 0.04 mg of | | | | | | |
| | SEQ ID | | | | | | |
| | NO: 2 | | | | | | |
| | variant | | | | | | |

The modification was done by adding the mentioned ingredient to the original detergent composition and mixing the ingredients on a magnetic stirrer for 15 min until the ingredients were totally dissolved and one homogenous solution was obtained.
The lipase variant used is a lipase variant of SEQ ID NO: 2 further with the following modifications D27R, G38A, D96E, D111A, G163K, D254S and P256T.

### Example 2

This is to test the effect of salt content in the described detergent composition on wash performance on greasy stains in a small-scale Terg-O-Tometer wash.

The detergent compositions are listed in the table 4, which were prepared based on adding salts to a commercial liquid detergent.

TABLE 3
swatch list

| | | Amount used in each |
|---|---|---|
| Producer | Swatch name | wash |
| Center For | KC-S-61 Beef fat with colorant | 2 pieces |
| Testmaterials | | |
| BV, | | |
| the Netherlands | | |

### Pre-Treatment Procedure of the Swatches

Pipette 0.5 g of each detergent in the table xx and drop it onto the stain area of each piece of swatch from table 3
Spread the detergent liquid over the stain area by one finger or a dropper bulb head
Leave for 5 min before regular TOM wash

### Terg-O-Tometer Wash Conditions

Temperature: 30° C.
Wash time: 20 min
Water level 1L
Water hardness: 14° dH, Ca²⁺:Mg²⁺:HCO₃⁻=2:1:4.5

### Equipment Used:

Washing machine: Terg-O-Tometer (TOM)
MacBeth Color Eye spectrophotometer
Mettler Toledo analytical balance AB104-S

### For the Preparation and Adjustment of Water Hardness the Following Ingredients are Needed:

Calcium chloride (CaCl₂.2H₂O)
Magnesium chloride (MgCL₂.6H₂O)
Sodium Hydrogen Carbonate (NaHCO₃)

### Ballast

The ballast weight, dryness and item composition must be the same in each wash.

After each wash the ballast is inactivated in an industrial washer at 95° C. /15 min wash without detergent.

### Drying Procedure and Measurement is Carried Out as Described in Example 1.

TABLE 4
results

| | | | | KC-S-61 |
|---|---|---|---|---|
| | | | KC-S-61 | Beef fat |
| | | | Beef fat | with |
| Detergent | Original | Modification to | with | colorant |
| used in | detergent | detergent | colorant | Delta |
| wash | composition | composition | R460 | R460 |
| 1 | Liby Quanxiao | none | 36.33 | |
| 2 | Liby Quanxiao | 0.08 mg of SEQ ID | 55.83 | 19.5 |
| | | NO: 2 variant | | |
| 3 | Liby Quanxiao | 2.5% sodium citrate | 43.47 | 7.14 |
| 4 | Liby Quanxiao | 2.5% sodium citrate, | 56.63 | 20.3 |
| | | 0.08 mg of SEQ ID | | |
| | | NO: 2 variant | | |
| 5 | Liby Quanxiao | 5% sodium citrate | 38.99 | 2.66 |
| 6 | Liby Quanxiao | 5% sodium citrate | 60.67 | 24.34 |
| | | 0.08 mg of SEQ ID | | |
| | | NO: 2 variant | | |
| 7 | Liby Quanxiao | 7.5% sodium citrate | 37.50 | 1.17 |
| 8 | Liby Quanxiao | 7.5% sodium citrate | 62.95 | 26.62 |
| | | 0.08 mg of SEQ ID | | |
| | | NO: 2 variant | | |
| 9 | Liby Quanxiao | 1.8% sodium sulfate | 48.85 | 12.52 |
| 10 | Liby Quanxiao | 1.8% sodium sulfate | 60.78 | 24.45 |
| | | 0.08 mg of SEQ ID | | |
| | | NO: 2 variant | | |
| 11 | Liby Quanxiao | 3.6% sodium sulfate | 41.77 | 5.44 |
| 12 | Liby Quanxiao | 3.6% sodium sulfate | 62.17 | 25.84 |
| | | 0.08 mg of SEQ ID | | |
| | | NO: 2 variant | | |
| 13 | Liby Quanxiao | 5.4% sodium sulfate | 39.36 | 3.03 |
| 14 | Liby Quanxiao | 5.4% sodium sulfate | 63.34 | 27.01 |
| | | 0.08 mg of SEQ ID | | |
| | | NO: 2 variant | | |

The modification was done by adding the mentioned ingredient to the original detergent composition and mixing the ingredients on a magnetic stirrer for 15 min until the ingredients were totally dissolved and one homogenous solution was obtained

The lipase variant used is a lipase variant of SEQ ID NO: 2 further with the following modifications D27R, G38A, D96E, D111A, G163K, D254S and P256T.

### Example 3

This is to test the effect of different kinds of salts in the described detergent composition on wash performance on greasy stains in a small-scale Terg-O-Tometer wash.

The detergent compositions are listed in the table 6, which were prepared based on adding salts to a commercial liquid detergent.

TABLE 5
swatch list

| | | Amount used in each |
|---|---|---|
| Producer | Swatch name | wash |
| Center For | KC-S-61 Beef fat with colorant | 2 pieces |
| Testmaterials | | |
| BV, | | |
| the Netherlands | | |

TABLE 6
results

| | | | | KC-S- |
|---|---|---|---|---|
| | | | KC-S- | 61 Beef |
| | | | 61 Beef | fat with |
| Detergent | | | fat with | colorant |
| used in | Original | Modification to | colorant | Delta |
| wash | detergent | detergent | R460 | R460 |
| 1 | Liby Quanxiao | none | 36.28 | |
| 2 | Liby Quanxiao | 0.08 mg of SEQ ID | 44.46 | 8.18 |
| | | NO: 2 variant | | |
| 3 | Liby Quanxiao | 2.8% calcium chloride | 42.40 | 6.12 |
| | | (MW: 110.99) | | |
| 4 | Liby Quanxiao | 2.8% calcium chloride, | 56.23 | 19.95 |
| | | 0.08 mg of SEQ ID | | |
| | | NO: 2 variant | | |
| 5 | Liby Quanxiao | 3.8% potassium chloride | 45.76 | 9.48 |
| | | (MW: 74.55) | | |
| 6 | Liby Quanxiao | 3.8% potassium chloride | 56.07 | 19.79 |
| | | 0.08 mg of SEQ ID | | |
| | | NO: 2 variant | | |
| 7 | Liby Quanxiao | 4.3% sodium bicarbonate | 36.86 | 0.58 |
| | | (MW: 84.01) | | |
| 8 | Liby Quanxiao | 4.3% sodium bicarbonate | 52.99 | 16.71 |
| | | 0.08 mg of SEQ ID | | |
| | | NO: 2 variant | | |

The lipase variant used is a lipase variant of SEQ ID NO: 2 further with the following modifications D27R, G38A, D96E, D111A, G163K, D254S and P256T.

1-5. (canceled)

6. A method for laundering a textile, comprising:
a. applying the liquid detergent composition of claim 7 on the textile,
b. leaving the detergent composition on the textile for less than 12 hours,
c. washing the textile in a wash liquor comprising the liquid detergent composition of claim 7; and
d. optionally rinsing the textile.

7. A liquid detergent composition, comprising:
a. a lipase in an amount of 0.025-0.6 mg enzyme protein/gram detergent composition,
b. a surfactant, and
c. a salt comprising a cation and a halogen or a cation and an organic anion, wherein the cation is monovalent, divalent or trivalent and the salt is present in a concentration in the range of 0.00005-0.004 mol/gram detergent composition,
wherein the ratio of lipase (mg enzyme protein/gram detergent composition) relative to water (g water/gram detergent composition) is in the range of 1:167 to 1:38000.

8. The composition according to claim 7, wherein the surfactant is in an amount of 0.1-0.6 g/gram detergent composition.

9. The composition according to claim 7, wherein the monovalent, divalent or trivalent cations are selected from the group consisting of sodium, potassium, magnesium, calcium, boron, aluminium, iron, copper, zinc and a mixture thereof.

10. The composition according to claim 7, wherein the organic anion is selected from the group consisting of formate, acetate, citrate, carbonate, hydrogen carbonate and lactate.

11. The composition according to claim 7, wherein the salt is sodium citrate, sodium chloride, sodium carbonate, potassium chloride, calcium chloride or a mixture thereof.

12. The composition according to claim 7, wherein the lipase has at least 80% sequence identity to SEQ ID NO: 1 or at least 80% sequence identity to SEQ ID NO: 2.

13. The composition according to claim 12, wherein the lipase is a variant of a parent lipase and is selected from the group consisting of:
a. variants having at least 85% sequence identity to SEQ ID NO: 1; and
b. variants having at 85% sequence identity to SEQ ID NO: 2.

14. The composition according to claim 12, wherein the lipase is a variant of a parent lipase, which variant has lipase activity, has at least 80% but less than 100% sequence identity with SEQ ID NO: 2, and comprises one or more of the following substitutions D96E, D111A, D254S, G163K, P256T, G91T and G38A wherein numbering is according to SEQ ID NO: 2.

15. The composition according to claim 14, wherein the variant further comprises substitutions at positions corresponding to D27R or N33Q of SEQ ID NO: 2.

16. The composition according to claim 15, wherein the variant comprises the substitutions D27R, G38A, D96E, D111A, G163K, D254S and P256T.


SEQUENCE LISTING (2 sequences)

SEQ ID NO: 1 - protein, 269 aa, Humicola lanuginosa [sequence omitted]

SEQ ID NO: 2 - protein, 269 aa, Thermomyces lanuginosus
EVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVRRRDIVKIEGIDATGGNNQPNIPDIPAHLWYFGLIGTCL